(19) United States
(12) Patent Application Publication
(10) Pub. No.: US 2025/0221860 A1
(43) Pub. Date: Jul. 10, 2025

(54) DEVICE, SYSTEM, AND METHOD FOR TINNITUS TREATMENT

(71) Applicant: NATIONAL CHENG KUNG UNIVERSITY, Tainan City (TW)

(72) Inventors: Chou-Ching LIN, Tainan City (TW); Chun-Chia YANG, Taichung City (TW); Chun-Wei TANG, Tainan City (TW)

(21) Appl. No.: 18/409,670

(22) Filed: Jan. 10, 2024

(51) Int. Cl. A61F 11/04

(52) U.S. Cl. CPC A61F 11/045

(57) ABSTRACT

A tinnitus treatment device includes a database module storing a Shepard scale that includes multiple Shepard tones, and a playback control module configured to repeatedly execute a loop playback procedure. In the loop playback procedure, the playback control module reads the Shepard scale from the database module, and outputs the Shepard scale multiple times to an audio output device in a manner that the Shepard tones are outputted in order from high frequency to low frequency. The playback control module stops output of the Shepard scale for a resting-time interval after each execution of the loop playback procedure, and a length of the resting-time interval is equal to time taken for a single execution of the loop playback procedure.

### FIELD

The disclosure relates to a treatment device and a treatment method, and more particularly to a tinnitus treatment device and a tinnitus treatment method.

### BACKGROUND

People experiencing tinnitus symptoms may hear annoying sounds that do not actually exist. The annoying sounds may vary, ranging from low and deep tones to high-pitched tones, and could be so loud that people may be distracted or unable to hear the sounds of the real world.

Due to the incomplete understanding of the mechanisms that cause tinnitus, current sound therapies for tinnitus are merely experimental and research-oriented approaches. These sound therapies may yield different outcomes for individuals with different tinnitus symptoms, which results in varying therapeutic effects.

### SUMMARY

Therefore, an object of the disclosure is to provide a tinnitus treatment device, a tinnitus treatment system, and a tinnitus treatment method.

According to the disclosure, the tinnitus treatment device is adapted for use with an audio output device to perform tinnitus treatment on a user who has tinnitus at a tinnitus frequency. The tinnitus treatment device includes a communication module that is to be communicatively connected to the audio output device, and a tinnitus treatment system that is communicatively connected to the communication module. The tinnitus treatment system includes a database module and a playback control module. The database module stores a Shepard scale that includes a plurality of Shepard tones and that spans a frequency range covering the tinnitus frequency. The playback control module includes a playback control unit that is communicatively connected to the database module, and that is configured to, when activated, execute a loop playback procedure T number of times, where T≥2. In the loop playback procedure, the playback control unit reads the Shepard scale from the database module, and outputs the Shepard scale S number of times to the audio output device through the communication module in a manner that the Shepard tones are outputted in order from high frequency to low frequency, where S≥2. The playback control unit is configured to stop output of the Shepard scale for a resting-time interval after each execution of the loop playback procedure, and a length of the resting-time interval is equal to time taken for a single execution of the loop playback procedure.

According to the disclosure, the tinnitus treatment system is implemented by at least one of an electronic circuit or a software program that is constructed within an electronic device, to perform tinnitus treatment on a user who has tinnitus at a tinnitus frequency. The tinnitus treatment system includes a database module and a playback control module. The database module stores a Shepard scale that includes a plurality of Shepard tones and that spans a frequency range covering the tinnitus frequency. The playback control module includes a playback control unit that is communicatively connected to the database module, and that is configured to, when activated, execute a loop playback procedure T number of times, where T≥2. In the loop playback procedure, the playback control unit reads the Shepard scale from the database module, and outputs the Shepard scale S number of times to an audio output device in a manner that the Shepard tones are outputted in order from high frequency to low frequency, where S≥2. The playback control unit is configured to stop output of the Shepard scale for a resting-time interval after each execution of the loop playback procedure, and a length of the resting-time interval is equal to time taken for a single execution of the loop playback procedure.

According to the disclosure, the tinnitus treatment method is adapted for a user who has tinnitus at a tinnitus frequency, and includes an establishing step and an outputting step. In the establishing step, a tinnitus treatment device establishes a Shepard scale that includes a plurality of Shepard tones and that spans a frequency range covering the tinnitus frequency. In the outputting step, the tinnitus treatment device executes a loop playback procedure T number of times, where T≥2. In the loop playback procedure, the tinnitus treatment device outputs the Shepard scale S number of times to an audio output device in a manner that the Shepard tones are outputted in order from high frequency to low frequency, where S≥2. In the outputting step, the tinnitus treatment device stops output of the Shepard scale for a resting-time interval after each execution of the loop playback procedure, and a length of the resting-time interval is equal to time taken for a single execution of the loop playback procedure.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating an embodiment of a tinnitus treatment device that is used with an audio output device according to the disclosure.

FIG. 2 is a block diagram illustrating the embodiment of the tinnitus treatment device according to the disclosure.

FIG. 3 is a flow chart illustrating an embodiment of a tinnitus treatment method according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

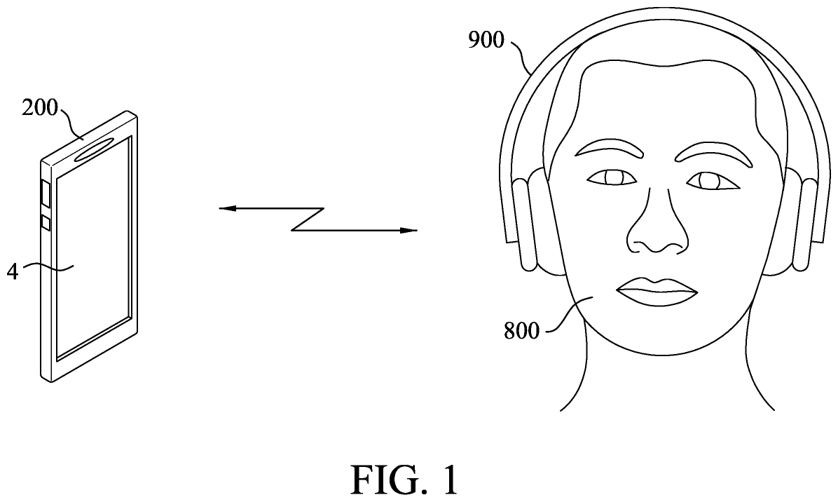

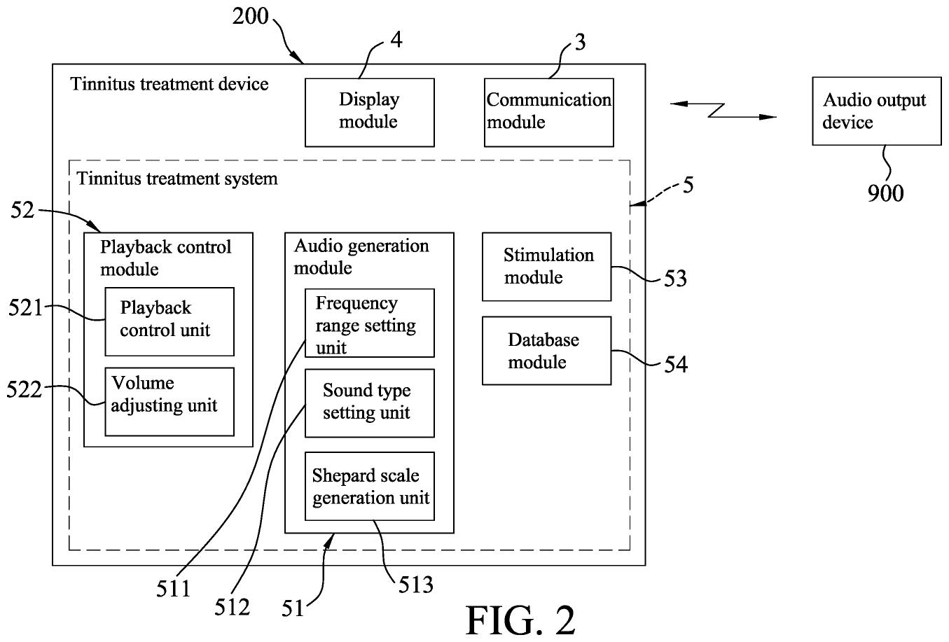

Referring to FIGS. 1 and 2, an embodiment of a tinnitus treatment device 200 according to this disclosure is adapted for use with an audio output device 900 to perform tinnitus treatment on a patient 800 who has a tinnitus symptom. The audio output device 900 is configured to transmit sounds for the patient 800 to listen to. In this embodiment, the audio output device 900 is exemplified as a headphone for the patient 800 to wear over the ears. In some embodiments, the audio output device 900 may be a headphone or an earphone that employs either air conduction or bone conduction, and this disclosure is not limited in this respect. In some embodiments, the audio output device 900 may be a loudspeaker. Since there is a wide diversity of audio output devices, and since the audio output device 900 is not a main focus of this disclosure, implementation of the audio output device 900 is not limited to the above-mentioned examples.

The tinnitus treatment device 200 includes a communication module 3, a display module 4, and a tinnitus treatment system 5 that is communicatively connected to the communication module 3 and the display module 4. In this embodiment, the communication module 3 is communicatively connected to the audio output device 900 through conventional wireless communication technology, which enables communication and data transmission with the audio output device 900. For example, the wireless communication technology may include Bluetooth®, but not limited thereto. In some embodiments, the communication module 3 may be communicatively connected to the audio output device 900 through signal wires. The communication module 3 comes in a wide variety of types, and further details are not provided herein for the sake of brevity. In this embodiment, the display module 4 is exemplified as a touchscreen display, such as but not limited to using a capacitive touchscreen, which allows touch-based operations for controlling operation of the tinnitus treatment system 5.

The tinnitus treatment system 5 is configured to output a Shepard scale to perform tinnitus treatment, and may be implemented through, for example, electronic circuits and software programs constructed within electronic components of an electronic device, such as but not limited to a microprocessor circuit with C programming language capabilities. The tinnitus treatment system 5 includes an audio generation module 51, a playback control module 52, a stimulation module 53, and a database module 54.

The database module 54 has a plurality of sound templates that correspond to different types of sounds (e.g., pure tones, narrowband noise, etc.) built therein, and may be realized as, for example but not limited to, read-only memory (ROM) such as programmable read-only memory (PROM) or erasable programmable read-only memory (EPROM), a hard disk drive (HDD), or a solid state drive (SSD), etc.

The audio generation module 51 includes a frequency range setting unit 511, a sound type setting unit 512, and a Shepard scale generation unit 513. The frequency range setting unit 511 is operable to input a frequency of a tinnitus sound heard by the patient 800 (hereinafter referred to as “tinnitus frequency”). For instance, the frequency range setting unit 511 may be a user interface to be displayed and operated through the display module 4, but this disclosure is not limited in this respect. In other words, the patient 800, who is a user of the tinnitus treatment device 200, has tinnitus at the tinnitus frequency. The tinnitus frequency can be determined by conducting pitch-matching tests on the patient 800 during tinnitus acoustic measurements, and the tinnitus frequency may vary among different patients. Since obtaining the tinnitus frequency for the patient 800 can be achieved using conventional medical diagnostic techniques and is not a focus of this disclosure, details thereof are omitted herein for the sake of brevity.

In this embodiment, the Shepard scale is composed of a plurality of different Shepard tones, and is established around or based on the tinnitus frequency to span a frequency range covering the tinnitus frequency, where the Shepard tones are equidistantly distributed on a logarithmic scale and are arranged in order from high frequency to low frequency (or from high pitch to low pitch). The frequency range may be from M number of perfect octaves lower than the tinnitus frequency to N number of perfect octaves higher than the tinnitus frequency, where each of M and N is a positive integer, and the values of M and N can be inputted by operating the frequency range setting unit 511. In cases where M=N, the Shepard scale may be established with the tinnitus frequency serving as a center frequency. In this embodiment, M=N=1, and the Shepard scale is a descending chromatic Shepard scale composed of twelve Shepard tones (i.e., for any consecutive two of the Shepard tones in the Shepard scale, the latter one is one semitone lower than the previous one in pitch), where the twelve Shepard tones consist of twenty-four pitches of different frequencies within two adjacent chromatic scales, but this disclosure is not limited in this respect.

The sound type setting unit 512 is communicatively coupled to the database module 54, and is operable to select, from the database module 54, one of the sound templates to be used to make the Shepard scale.

The Shepard scale generation unit 513 is communicatively coupled to the frequency range setting unit 511, the sound type setting unit 512, and the database module 54, and is configured to perform sound synthesis through a sound processing program (e.g., Python or other programs capable of performing sound synthesis) to process the selected one of the sound templates (received from the sound type setting unit 512) based on the tinnitus frequency (received from the frequency range setting unit 511) and the values of M and N (received from the frequency range setting unit 511), so as to generate the Shepard scale, and then store the Shepard scale into the database module 54. The Shepard scale generation unit 513 further performs sound synthesis based on the tinnitus frequency and the selected one of the sound templates to generate a stimulus sound, and stores the stimulus sound into the database module 54 in, for example, Waveform Audio File (WAV) format.

The playback control module 52 includes a playback control unit 521 and a volume adjusting unit 522. The playback control unit 521 is built-in with a loop playback mode. When the tinnitus treatment system 5 is activated to perform tinnitus treatment, the playback control unit 521 enters the loop playback mode and executes a loop playback procedure T number of times, where T≥2. In the loop playback procedure, the playback control unit 521 reads the Shepard scale from the database module 54, and outputs the Shepard scale S number of times to the audio output device 900 through the communication module 3, where S≥2, and the audio output device 900 audibly outputs the Shepard scale for the patient 800 to listen to. In this embodiment, the playback control unit 521 outputs the Shepard scale in a manner that the Shepard tones of the Shepard scale are outputted in order from high frequency to low frequency at an interval of X milliseconds, where 450≤X≤500. Namely, the playback control unit 521 outputs the Shepard scale in the order from the highest Shepard tone to the lowest Shepard tone, and an interval between the outputs of two successive Shepard tones ranges from 450 milliseconds to 500 milliseconds. In some embodiments, the audible output of each Shepard tone lasts for the entire interval until the output of the next Shepard tone. Furthermore, in this embodiment, the playback control unit 521 is configured to repeatedly execute the loop playback procedure in a manner that the playback control unit 521 stops output of the Shepard scale for a resting-time interval after each execution of the loop playback procedure, and a length of the resting-time interval is equal to time taken for a single execution of the loop playback procedure (i.e., equaling time taken to output the Shepard scale S number of times).

The volume adjusting unit 522 is operable to adjust volume of the Shepard scale that is outputted by the playback control unit 521 to the audio output device 900 through the communication module 3 when the playback control unit 521 is executing tinnitus treatment.

The stimulation module 53 is simultaneously triggered when the playback control module 52 is activated to perform tinnitus treatment. During the repeated execution of the loop playback procedure, the stimulation module 53 randomly selects one or more of the resting-time intervals that are respectively after the T number of executions of the loop playback procedure, and outputs, to the audio output device 900 via the communication module 3, the stimulus sound for a predetermined period within the selected one(s) of the resting-time intervals. In this embodiment, the predetermined period ranges, for example, from eight seconds to twelve seconds, but this disclosure is not limited in this respect.

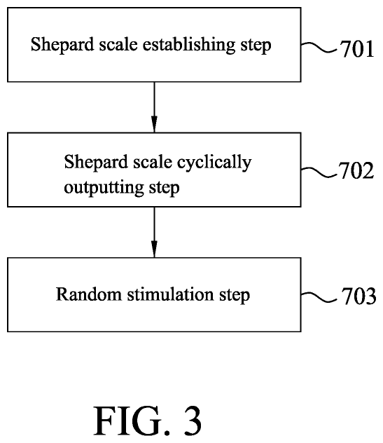

Further referring to FIG. 3, an embodiment of a tinnitus treatment method according to this disclosure is implemented by the tinnitus treatment device 200, and includes a Shepard scale establishing step 701, a Shepard scale cyclically outputting step 702, and a random stimulation step 703.

In the Shepard scale establishing step 701, the tinnitus treatment device 200 is operated to generate a Shepard scale and a stimulus sound based on a tinnitus frequency of the patient 800 which is acquired through medical examinations, where the Shepard scale spans a frequency range from M number of perfect octaves lower than the tinnitus frequency to N number of perfect octaves higher than the tinnitus frequency, and the frequency range covers the tinnitus frequency. In this embodiment, M=N=1, the tinnitus frequency is made to be a center frequency of the Shepard scale, and the stimulus sound is made to have the tinnitus frequency.

In the Shepard scale cyclically outputting step 702, the tinnitus treatment device 200 is operated to activate the tinnitus treatment function, and thus enters the loop playback mode to execute the loop playback procedure T number of times, where T≥2. In each execution of the loop playback procedure, the tinnitus treatment device 200 reads and outputs the Shepard scale S number of times to the audio output device 900 through the communication module 3 in a manner that the Shepard tones are read and outputted in order from high frequency to low frequency at an interval of X milliseconds, where S≥2, 450≤X≤500, and any two of the Shepard tones of the Shepard scale that are consecutively outputted differ from each other by one semitone. During the T number of executions of the loop playback procedure, the tinnitus treatment device 200 stops output of the Shepard scale for a resting-time interval after each execution of the loop playback procedure, and a length of the resting-time interval is equal to time taken for a single execution of the loop playback procedure.

In this embodiment, the playback control unit 521 reads and outputs the Shepard scale two times in each execution of the loop playback procedure, and the Shepard scale includes twelve different Shepard tones arranged from high frequency to low frequency. The playback control unit 521 outputs the Shepard tones one by one, and changes from one Shepard tone to another one every 500 ms, so a single execution of the loop playback procedure takes 12 seconds, and the resting-time interval is 12 seconds. If each session of tinnitus treatment lasts for thirty minutes, the number of executions of the loop playback procedure is approximately seventy-five (i.e., T=75).

In the random stimulation step 703, the tinnitus treatment device 200 randomly selects one or more of the resting-time intervals that are respectively after the T number of executions of the loop playback procedure, and outputs the stimulus sound with the tinnitus frequency for a predetermined period (e.g., ranging from eight seconds to twelve seconds) within the selected one(s) of the resting-time intervals.

Regarding tinnitus, one hypothesis in the research considers tinnitus as a type of illusion, which may arise from improper generation of an upward prediction error by lower-level neurons, which is passed on to higher-level neurons. When receiving the prediction error, the higher-level neurons have to generate a downward wrong prediction and pass the same to the lower-level neurons so as to make the upward and downward signals match to complete feedback control, and this wrong prediction is perceived as tinnitus.

This embodiment synthesizes and generates the Shepard scale and the stimulus sound based on the tinnitus frequency of the patient 800, and cyclically outputs the Shepard scale from high-frequency Shepard tones to low-frequency Shepard tones for the patient 800 to listen to, thereby giving the patient 800 the illusion of hearing sounds with continuously decreasing frequencies. When the output of the Shepard scale stops, the patient's brain would anticipate a continuation of descending tones, which induces a gradual decrease in the tinnitus frequency.

Furthermore, when the stimulus sound with the tinnitus frequency is heard unexpectedly during the resting-time interval after the patient 800 has been listening to the Shepard scale, it may trigger a conflict in perceptual experience. The conflict can be utilized to attenuate or modify the aforesaid prediction error between neurons, which contributes to improvement of tinnitus symptoms.

In one experiment, forty patients with tinnitus were enrolled as participants for a tinnitus treatment trial to verify the effects of the embodiment of the tinnitus treatment device 200. Each patient was assessed and diagnosed with tinnitus by an otolaryngologist. Criteria for selecting the participants are outlined in Table 1.

TABLE 1
Criteria

| | |
|---|---|
| 1 | Aged 20 or above |
| 2 | Suffering from subjective tinnitus and bilateral tinnitus |
| 3 | Tinnitus lasting for more than six months |
| 4 | No mental, emotional, or health-related disorders that would preclude |
| | the patient from participating in the study |
| 5 | No history of neurological or cognitive disorders |
| 6 | No self-perceived hearing impairments or barriers to communication |
| 7 | Able to comprehend the questionnaire and provide clear responses |
| 8 | Excluding those with Ménière's disease, acoustic neuroma, or other |
| | external or middle ear disorders |
| 9 | Excluding those with objective tinnitus or hyperacusis |

The forty participants were randomly assigned to an experimental group and a control group, with twenty participants in each group.

For each participant in the experimental group, the Shepard scale and the stimulus sound for the tinnitus treatment were customized based on their tinnitus frequency as described above (i.e., the Shepard scale being made to have a center frequency at the tinnitus frequency, and the stimulus sound being made to have the tinnitus frequency), where M=1, N=1, S=2, X=500, and the resting-time interval was 12 seconds. A tinnitus treatment duration for each participant was two weeks, with a daily treatment period of one hour, which was split into two sessions of half an hour each.

For each participant in the control group, the Shepard scale for the tinnitus treatment was customized to have a center frequency lower than their tinnitus frequency by a perfect octave, and the stimulus sound for the tinnitus treatment was customized to have the tinnitus frequency. The manner in which the stimulus sound was provided to the control group was the same as that for the experimental group.

Before and after the experiment, all participants from the experimental group and the control group were asked to complete a tinnitus handicap inventory (THI). The THI includes three subscales for assessment in functional, emotional, and severity aspects, and consists of twenty-five items, with response options being “frequently” (4 points), “sometimes” (2 points), and “never” (0 points), which yields a total score ranging from 0 to 100 points. THI scores are used to evaluate the degree of tinnitus-related impairment and the effect of sound therapy. A THI score exceeding 18 points is considered indicative of meeting referral criteria for intervention, so a threshold of “THI score≥18 points” is employed in experimental data presented below.

Since the THI is a conventional clinical tool used to assess the degree of tinnitus-related impairment, further details are omitted herein for the sake of brevity.

Among those participants with pre-treatment THI scores of 18 points or above, which include 11 participants from the experimental group and 14 participants from the control group, a two-factor mixed-design analysis of variance (ANOVA) was conducted to examine effect of the treatment methods in the experimental group and the control group on THI scores. The analysis results indicated a significant interaction between treatment methods and treatment duration on the THI scores, where F (1, 23)=5.67, and p=0.02.

Referring to Table 2, among those participants with pre-treatment THI scores of 18 points or above, a Wilcoxon signed-rank test was used to compare the change in THI scores before and after treatment. The analysis results indicated significant differences in THI scores before and after treatment in the experimental group (p=0.007), while no significant difference was shown in the control group (p=0.55).

TABLE 2

| | Pre-treatment THI Score ≥ 18 points | |
|---|---|---|
| THI Score | Experimental group: 11 ppl. | Control group: 14 ppl. |
| Standard Deviation | 6.98 | 7.26 |
| (SD) | | |
| P value | 0.007 | 0.55 |

Among the participants with pre-treatment THI scores of 18 points or above, a Mann-Whitney U test was used for analysis. The analysis results indicated a significant difference in the change of THI scores before and after treatment between the experimental group and the control group (p=0.02). In further detail, the experimental group had a greater decrease in THI scores compared to the control group, where the experimental group had an average decrease of 6 points in THI scores, and the control group had an average increase of 0.85 points in THI scores.

The average post-treatment changes in tinnitus frequency for those participants with pre-treatment THI scores of 18 points or above in the experimental group and the control group are presented in Table 3.

TABLE 3

| Experimental Group | | | Control Group | | |
|---|---|---|---|---|---|
| | Difference in tinnitus | | | Difference in tinnitus | |
| | frequency before and | | | frequency before and | |
| Participant | after treatment | | Participant | after treatment | |
| No. | Hz Scale | mel Scale | No. | Hz Scale | mel Scale |
| 1 | 888.00 | 626.23 | 0 | 397.00 | 85.63 |
| 4 | 3359.00 | 959.85 | 1 | 2919.00 | 626.00 |
| 5 | −76.00 | −50.04 | 2 | 80.00 | 12.09 |
| 8 | −2843.00 | −1178.26 | 3 | 2054.00 | 551.07 |
| 9 | −1587.00 | −218.62 | 8 | 426.00 | 92.15 |
| 10 | 2165.00 | −703.02 | 9 | −153.00 | −108.29 |
| 12 | −4737.00 | −556.84 | 10 | −58.00 | −21.29 |
| 13 | −1081.00 | −177.64 | 11 | −564.00 | −89.18 |
| 14 | −4809.00 | −715.2 | 12 | 473.00 | 87.50 |
| 17 | −296.00 | −215.6 | 13 | 94.00 | 85.27 |
| 18 | 1080.00 | 177.48 | 14 | −563.00 | −89.03 |
| | | | 16 | 1287.00 | 180.42 |
| | | | 17 | 209.00 | 124.32 |
| | | | 19 | 0.00 | 0.00 |
| Average | −721.55 | −58.78 | Average | 471.50 | 109.76 |
| Standard | 2638.69 | 643.57 | Standard | 983.21 | 220.81 |
| Deviation | | | Deviation | | |
| (SD) | | | (SD) | | |

It is noted that, in this embodiment, the tinnitus treatment system 5 may be an electronic component that is constructed in an electronic device and that is implemented using electronic circuits and software programs, but in other embodiments, the tinnitus treatment system 5 may be implemented as a software program available for download and installation on an electronic device, thereby enabling the electronic device installed with the tinnitus treatment system 5 to be used for tinnitus treatment. The software program may be, for example but not limited to, mobile application software that can be installed in mobile devices running on Android or iOS systems.

In summary, the tinnitus treatment system 5 is configured to enable the tinnitus treatment device 200 to cyclically output the Shepard scale, thereby giving the patient 800 the illusion of hearing sounds with continuously decreasing frequency, so that the patient 800 would anticipate a continuation of descending tones when the output of the Shepard scale is stopped, thereby inducing a gradual decrease of the tinnitus frequency. Furthermore, by randomly introducing the stimulus sound during a resting-time interval between two consecutive executions of the loop playback procedure, an unpredicted conflict may be created between the anticipated descending tones and the stimulus sound, and experimental evidence shows that the method of this disclosure is effective in treating tinnitus.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A tinnitus treatment device adapted for use with an audio output device to perform tinnitus treatment on a user who has tinnitus at a tinnitus frequency, said tinnitus treatment device comprising:
a communication module that is to be communicatively connected to the audio output device; and
a tinnitus treatment system that is communicatively connected to said communication module, and that includes:
a database module storing a Shepard scale that includes a plurality of Shepard tones and that spans a frequency range covering the tinnitus frequency; and
a playback control module including a playback control unit that is communicatively connected to said database module, and that is configured to, when activated, execute a loop playback procedure T number of times, where T≥2;
wherein, in the loop playback procedure, said playback control unit reads the Shepard scale from said database module, and outputs the Shepard scale S number of times to the audio output device through said communication module in a manner that the Shepard tones are outputted in order from high frequency to low frequency, where S≥2; and
wherein said playback control unit is configured to stop output of the Shepard scale for a resting-time interval after each execution of the loop playback procedure, and a length of the resting-time interval is equal to time taken for a single execution of the loop playback procedure.

2. The tinnitus treatment device as claimed in claim 1, wherein in the loop playback procedure, the Shepard tones are outputted at an interval of X milliseconds, where 450≤X≤500.

3. The tinnitus treatment device as claimed in claim 1, wherein any two of the Shepard tones of the Shepard scale that are consecutively outputted differ from each other by one semitone.

4. The tinnitus treatment device as claimed in claim 3, wherein the Shepard scale spans the frequency range from one perfect octave lower than the tinnitus frequency to one perfect octave higher than the tinnitus frequency, and consists of twenty-four pitches of different frequencies.

5. The tinnitus treatment device as claimed in claim 1, wherein said database module has a plurality of sound templates built therein, and said tinnitus treatment system includes an audio generation module;
wherein said audio generation module includes:
a frequency range setting unit that is operable to input the tinnitus frequency;
a sound type setting unit that is operable to select one of the sound templates from said database module; and
a Shepard scale generation unit that is configured to process the selected one of the sound templates into the Shepard scale based on the tinnitus frequency, and to store the Shepard scale into said database module.

6. The tinnitus treatment device as claimed in claim 1, wherein said tinnitus treatment system includes a stimulation module that is configured to randomly select one of the resting-time intervals that are respectively after the T number of executions of the loop playback procedure, and to output, to the audio output device via said communication module, a stimulus sound for a predetermined period within the selected one of the resting-time intervals.

7. The tinnitus treatment device as claimed in claim 6, wherein the predetermined period for which the stimulus sound is outputted ranges from eight seconds to twelve seconds.

8. The tinnitus treatment device as claimed in claim 1, wherein said playback control module includes a volume adjusting unit operable to adjust volume of the Shepard scale that is outputted by said playback control unit to the audio output device through said communication module.

9. A tinnitus treatment system, implemented by at least one of an electronic circuit or a software program that is constructed within an electronic device, to perform tinnitus treatment on a user who has tinnitus at a tinnitus frequency, said tinnitus treatment system comprising:
a database module storing a Shepard scale that includes a plurality of Shepard tones and that spans a frequency range covering the tinnitus frequency; and
a playback control module including a playback control unit that is communicatively connected to said database module, and that is configured to, when activated, execute a loop playback procedure T number of times, where T≥2;
wherein, in the loop playback procedure, said playback control unit reads the Shepard scale from said database module, and outputs the Shepard scale S number of times to an audio output device in a manner that the Shepard tones are outputted in order from high frequency to low frequency, where S≥2; and
wherein said playback control unit is configured to stop output of the Shepard scale for a resting-time interval after each execution of the loop playback procedure, and a length of the resting-time interval is equal to time taken for a single execution of the loop playback procedure.

10. The tinnitus treatment system as claimed in claim 9, wherein, in the loop playback procedure, the Shepard tones are outputted at an interval of X milliseconds, where 450≤X≤500.

11. The tinnitus treatment system as claimed in claim 9, wherein any two of the Shepard tones of the Shepard scale that are consecutively outputted differ from each other by one semitone.

12. The tinnitus treatment system as claimed in claim 11, wherein the Shepard scale spans the frequency range from one perfect octave lower than the tinnitus frequency to one perfect octave higher than the tinnitus frequency, and consists of twenty-four pitches of different frequencies.

13. The tinnitus treatment system as claimed in claim 9, wherein said database module has a plurality of sound templates built therein, and said tinnitus treatment system further comprises an audio generation module that includes:
a frequency range setting unit that is operable to input the tinnitus frequency;
a sound type setting unit that is operable to select one of the sound templates from said database module; and
a Shepard scale generation unit that is configured to process the selected one of the sound templates into the Shepard scale based on the tinnitus frequency, and to store the Shepard scale into said database module.

14. The tinnitus treatment system as claimed in claim 9, further comprising a stimulation module that is configured to randomly select one of the resting-time intervals that are respectively after the T number of executions of the loop playback procedure, and to output to the audio output device a stimulus sound for a predetermined period within the selected one of the resting-time intervals.

15. The tinnitus treatment system as claimed in claim 14, wherein the predetermined period for which the stimulus sound is outputted ranges from eight seconds to twelve seconds.

16. A tinnitus treatment method adapted for a user who has tinnitus at a tinnitus frequency, comprising:
an establishing step, in which a tinnitus treatment device establishes a Shepard scale that includes a plurality of Shepard tones and that spans a frequency range covering the tinnitus frequency; and
an outputting step, in which the tinnitus treatment device executes a loop playback procedure T number of times, where T≥2;
wherein, in the loop playback procedure, the tinnitus treatment device outputs the Shepard scale S number of times to an audio output device in a manner that the Shepard tones are outputted in order from high frequency to low frequency, where S≥2; and
wherein, in the outputting step, the tinnitus treatment device stops output of the Shepard scale for a resting-time interval after each execution of the loop playback procedure, and a length of the resting-time interval is equal to time taken for a single execution of the loop playback procedure.

17. The tinnitus treatment method as claimed in claim 16, wherein in the loop playback procedure, the Shepard tones are outputted at an interval of X milliseconds, where 450≤X≤500.

18. The tinnitus treatment method as claimed in claim 16, wherein any two of the Shepard tones of the Shepard scale that are consecutively outputted differ from each other by one semitone.

19. The tinnitus treatment method as claimed in claim 18, wherein the Shepard scale spans the frequency range from one perfect octave lower than the tinnitus frequency to one perfect octave higher than the tinnitus frequency, and consists of twenty-four pitches of different frequencies.

20. The tinnitus treatment method as claimed in claim 16, further comprising a stimulation step, in which the tinnitus treatment device randomly selects one of the resting-time intervals that are respectively after the T number of executions of the loop playback procedure, and outputs to the audio output device a stimulus sound for a predetermined period ranging from eight seconds to twelve seconds within the selected one of the resting-time intervals.
